(19) United States
(12) Patent Application Publication
(10) Pub. No.: US 2020/0030561 A1
(43) Pub. Date: Jan. 30, 2020

(54) KITS AND METHODS FOR RETROGRADE PERCUTANEOUS DILATIONAL TRACHEOSTOMY

(71) Applicant: Hamad Medical Corporation, Doha (QA)

(72) Inventor: Amr Salah Ibrahim Elsid Omar, Doha (QA)

(21) Appl. No.: 16/593,644

(22) Filed: Oct. 4, 2019

Related U.S. Application Data
(63) Continuation-in-part of application No. PCT/US2018/026174, filed on Apr. 5, 2018.
(60) Provisional application No. 62/483,317, filed on Apr. 7, 2017.
(60) Provisional application No. 62/744,999, filed on Oct. 12, 2018.

(51) Int. Cl. A61M 16/04

(52) U.S. Cl. CPC A61M 16/0472; A61B 1/2676; A61M 16/0488; A61M 16/045

(57) ABSTRACT

The present disclosure provides for a method of performing a percutaneous dilational tracheostomy (“PDT”) that includes inserting a tracheal tube including an inflatable cuff through a mouth and into a trachea of a patient; inserting a bronchoscope into the tracheal tube; inserting a puncturing wire through a channel of the bronchoscope. The puncturing wire includes a sharp cutting end and a dull gripping end. The method also includes puncturing the trachea of the patient with the sharp cutting end of the puncturing wire from within the trachea at a selected location to create a stoma; passing at least one dilator over the sharp cutting end of the puncturing wire for dilating the stoma; inserting a tracheostomy tube into the trachea through the stoma; and removing the puncturing wire from the patient.

### PRIORITY CLAIM

The present application is a continuation-in-part of PCT Patent Application No. PCT/US2018/026174 filed on Apr. 5, 2018, which claims priority to and the benefit of U.S. Provisional Application 62/483,317 filed on Apr. 7, 2017. The present application further claims priority to and the benefit of U.S. Provisional Application 62/744,999 filed on Oct. 12, 2018. The entirety of each are incorporated herein by reference.

### BACKGROUND

Managing patient airways remains a major concern in intensive care units (“ICU”) for patients that need assistance breathing. A tracheostomy is a medical procedure for providing an air passage to help a patient breathe when the usual rout for breathing is obstructed or impaired. The tracheostomy procedure may provide certain benefits. For example, patient comfort may be an immediate benefit because sedation and analgesic demands may be significantly reduced. In another example, the tracheostomy procedure is associated with improving the ability to wean a patient from mechanical ventilation, and thus can decrease the length of the patient's ICU stay.

A tracheostomy procedure may either be an operative tracheostomy or a percutaneous tracheostomy. An operative tracheostomy procedure is a surgical procedure that involves dissection and incision of the trachea under direct vision. A percutaneous dilational tracheostomy (“PDT”), also referred to as a bedside tracheostomy, is the placement of a tracheostomy tube without direct surgical visualization of the trachea, and is a minimally invasive procedure that may be easily performed in the ICU or at the patient's bedside. Accordingly, a PDT offers numerous advantages compared to an operative tracheostomy. For instance, it requires less time to perform a PDT, it is less expensive, and it may typically be performed sooner because an operating room is not required. Additionally, overall complications may be less frequent for PDT when compared to operative tracheostomies.

However, PDT has an increased risk of anterior tracheal injury and posterior tracheal wall perforation. The major complications encountered from PDT are bleeding, false track, esophageal perforation, pneumothorax, and conversion to surgical tracheostomy. The minor complications may include bleeding, subcutaneous emphysema, air leakage cuff, puncturing of the endotracheal tube or posterior tracheal wall, accidental detubation, and hypotension. Accordingly, the PDT procedure may be improved.

### BRIEF SUMMARY OF THE INVENTION

The present disclosure generally relates to a percutaneous dilational tracheostomy (“PDT”). In particular, the present disclosure provides a kit and method for performing a PDT including puncturing a patient's trachea from within the patient's trachea with a puncturing wire that includes an inner core and an outer sheath.

In light of the disclosures herein, and without limiting the scope of the invention in any way, in a first aspect of the present disclosure, which may be combined with any other aspect listed herein unless specified otherwise, a method of performing a percutaneous dilational tracheostomy (“PDT”) includes inserting a tracheal tube including an inflatable cuff through a mouth and into a trachea of a patient; inserting a bronchoscope into the tracheal tube; inserting a puncturing wire through a channel of the bronchoscope. The puncturing wire includes a sharp cutting end opposite a dull gripping end. The method also includes puncturing the trachea of the patient with the sharp cutting end of the puncturing wire from within the trachea at a selected location to create a stoma; passing at least one dilator over the sharp cutting end of the puncturing wire for dilating the stoma; inserting a tracheostomy tube into the trachea through the stoma; and removing the puncturing wire from the patient.

In a second aspect of the present disclosure, which may be combined with any other aspect listed herein unless specified otherwise, the method further includes pulling the sharp cutting end of the puncturing wire once the sharp cutting end passes through at least the trachea or skin of the patient. The pulling advances a portion of the puncturing wire out of the patient.

In a third aspect of the present disclosure, which may be combined with any other aspect listed herein unless specified otherwise, the puncturing wire includes multiple markings, and the sharp cutting end is pulled until at least one of the multiple markings appears outside of the patient.

In a fourth aspect of the present disclosure, which may be combined with any other aspect listed herein unless specified otherwise, the method further includes manipulating the puncturing wire once a marking nearest the dull gripping end of the multiple markings appears outside of the patient, wherein the manipulating includes positioning the puncturing wire such that the dull gripping end faces a tracheal bifurcation of the patient.

In a fifth aspect of the present disclosure, which may be combined with any other aspect listed herein unless specified otherwise, the method further includes, after the puncturing wire is manipulated, advancing the puncturing wire into the patient until the next adjacent marking to the marking nearest the dull gripping end is near the skin of the patient.

In a sixth aspect of the present disclosure, which may be combined with any other aspect listed herein unless specified otherwise, the selected location is between the second and third tracheal rings of the patient.

In a seventh aspect of the present disclosure, which may be combined with any other aspect listed herein unless specified otherwise, puncturing the trachea of the patient includes providing support to the sharp cutting end with the bronchoscope when the sharp cutting end punctures the trachea.

In an eighth aspect of the present disclosure, which may be combined with any other aspect listed herein unless specified otherwise, the puncturing wire includes an inner core at least partially enclosed by an outer sheath. The inner core includes the sharp cutting end, and the outer sheath includes the dull gripping end opposite the sharp cutting end.

In a ninth aspect of the present disclosure, which may be combined with any other aspect listed herein unless specified otherwise, the method further includes inflating the inflatable cuff of the tracheal tube at a position just below the vocal chords of the patient.

In a tenth aspect of the present disclosure, which may be combined with any other aspect listed herein unless specified otherwise, a kit for performing a PDT includes a bronchoscope including a channel, a tracheal tube, and a puncturing wire. The tracheal tube includes an insertion end and a free end with an inflatable cuff near the insertion end, and the tracheal tube is adapted to receive the bronchoscope. The puncturing wire includes a sharp cutting end and a dull gripping end, and is adapted to be removably inserted in the channel of the bronchoscope. The sharp cutting end is adapted to cut a stoma through a trachea of a patient from within the trachea at a selected site.

In an eleventh aspect of the present disclosure, which may be combined with any other aspect listed herein unless specified otherwise, the kit further includes at least one stomal dilator and a tracheostomy tube.

In a twelfth aspect of the present disclosure, which may be combined with any other aspect listed herein unless specified otherwise, the at least one stomal dilator includes a 14-French dilator.

In a thirteenth aspect of the present disclosure, which may be combined with any other aspect listed herein unless specified otherwise, the puncturing wire is coated in a friction-reducing material.

In a fourteenth aspect of the present disclosure, which may be combined with any other aspect listed herein unless specified otherwise, the friction-reducing material includes polytetrafluoroethylene.

In a fifteenth aspect of the present disclosure, which may be combined with any other aspect listed herein unless specified otherwise, the puncturing wire has a length between approximately 90 cm and approximately 1.5 m.

In a sixteenth aspect of the present disclosure, which may be combined with any other aspect listed herein unless specified otherwise, the puncturing wire includes at least one marking.

In a seventeenth aspect of the present disclosure, which may be combined with any other aspect listed herein unless specified otherwise, each of the at least one marking is a color or an indentation.

In a eighteenth aspect of the present disclosure, which may be combined with any other aspect listed herein unless specified otherwise, the puncturing wire includes multiple markings including a first marking positioned approximately 5 cm from the dull gripping end, a second marking positioned approximately 15 cm from the dull gripping end, and a third marking positioned approximately 20 cm from the dull gripping end.

In a nineteenth aspect of the present disclosure, which may be combined with any other aspect listed herein unless specified otherwise, the first marking is a first color, the second marking is a second color, and the third marking is a third color, and wherein the first color is different than at least one of the second color and the third color.

In a twentieth aspect of the present disclosure, which may be combined with any other aspect listed herein unless specified otherwise, the puncturing wire includes an inner core at least partially enclosed by an outer sheath. The inner core includes the sharp cutting end, and the outer sheath includes the dull gripping end opposite the sharp cutting end.

### BRIEF DESCRIPTION OF THE FIGURES

FIGS. 1A to 1C illustrate schematic diagrams of puncturing wires, according to an aspect of the present disclosure.

FIG. 2 illustrates a tracheal tube inserted into a patient, according to an aspect of the present disclosure.

FIG. 3 illustrates a bronchoscope advanced through the tracheal tube of FIG. 2, according to an aspect of the present disclosure.

FIG. 4 illustrates a puncturing wire advanced through a channel of the bronchoscope of FIG. 3 after puncturing the patient's trachea, according to an aspect of the present disclosure.

FIG. 5 illustrates an example method for performing a percutaneous dilational tracheostomy, according to an aspect of the present disclosure.

FIG. 6 illustrates an example kit for performing a percutaneous dilational tracheostomy, according to an aspect of the present disclosure.

### DETAILED DESCRIPTION

The present disclosure generally relates to a percutaneous dilational tracheostomy (“PDT”), also referred to as a bedside tracheostomy. In particular, the present disclosure provides a kit and method for performing a PDT including puncturing a patient's trachea from within the patient's trachea with a puncturing wire that includes a sharp cutting end and a dull gripping end. Early tracheostomy procedures reduce the incidence and severity of laryngeal injuries and sinusitis, improve the weaning of patient's off of mechanical ventilation, and shorten a patient's ICU stay. Thus, early tracheostomy using the disclosed kits and methods can allow medical professionals to quickly obtain these benefits to decrease the length of a patient's overall hospital stay. Accordingly, by decreasing patients' ICU stays and overall hospital stays, hospital resources such as beds become available more quickly for incoming patients.

Additionally, the presently disclosed methods require less training and may minimize the experience needed to perform the procedure with the disclosed kit. Moreover, the disclosed methods may be performed by two medical professionals instead of three, as required when performing a conventional PDT. Further, the disclosed kits and methods may decrease the PDT procedure time and decrease the risk of stoma misplacement, false track, puncturing the posterior of the trachea or the tracheal tube, and of components being entrapped in the tracheal tube. Accordingly, the present disclosure may improve a medical facility's quality of care in a cost-effective manner by decreasing the incidence of tracheostomy complications, decreasing the tracheostomy procedure time, and reducing patients' post-procedural ICU stay.

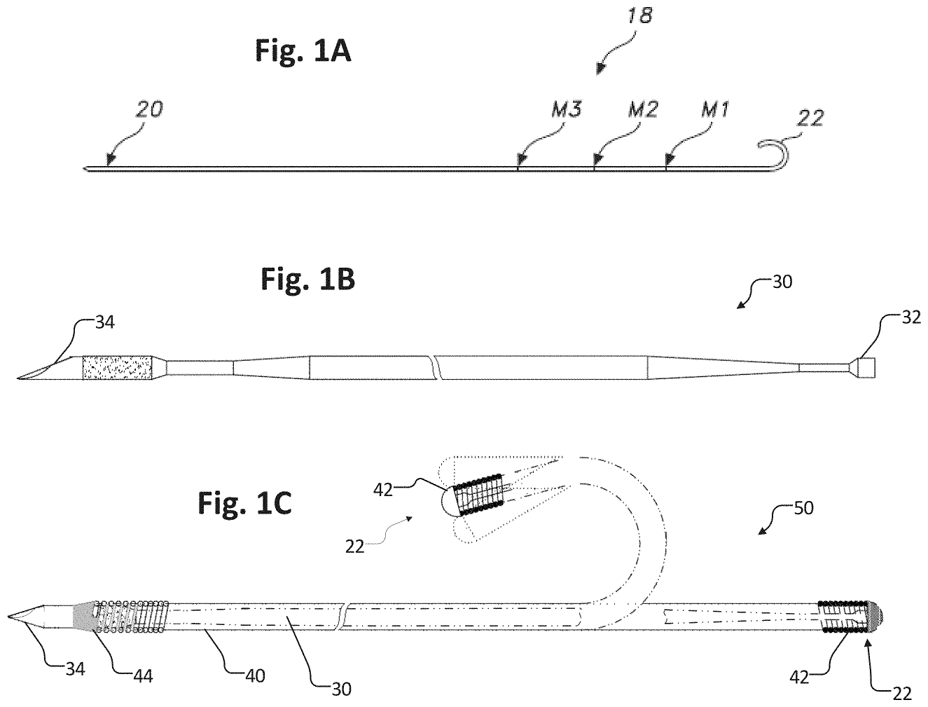

Before describing an example PDT procedure utilizing the provided kits and methods, the flexible puncturing wire 18 component will be described. As depicted in FIG. 1A, the puncturing wire 18 may have a cutting end 20 and a gripping end 22. The cutting end 20 of the puncturing wire 18 is sharp and defines a cutting end. For instance, the sharp cutting end 20 may be sharpened in three dimensions such that it is adapted to puncture a patient's trachea and skin from within the patient's trachea and advance through to reach the environment outside of the patient. The gripping end 22 is adapted to be gripped by a medical professional. In some instances, the gripping end 22 may be flexible, curved and/or dull. The puncturing wire 18 may be any suitable length for performing a tracheostomy procedure. In some examples, the puncturing wire 18 is between about 90 cm and about 1.5 m in length. In some examples, the puncturing wire 18 may be coated with a layer of friction-reducing material so that the puncturing wire 18 may more easily slide through a bronchoscope channel and/or the insides of a patient (e.g., the patient's air passage). For instance, the friction-reducing material may be polytetrafluoroethylene.

In various examples of the present disclosure, the puncturing wire 18 may have one or more markings, such as markings M1, M2, and M3 depicted in FIG. 1A. In other examples, the puncturing wire 18 may have more or less markings. The markings on the puncturing wire 18 may indicate to a medical professional performing the PDT procedure that the puncturing wire 18 is in a proper position to perform the next step in the procedure. Accordingly, the one or more markings may be at specific locations on the puncturing wire 18, such as certain distances away from the cutting end 20 or the gripping end 22 of the puncturing wire 18. For instance, in one example, the marking M1 is at a point 5 cm from the gripping end 22 of the puncturing wire 18, the marking M2 is at a point 15 cm from the gripping end 22, and the marking M3 is at a point 20 cm from the gripping end 22.

The markings may be any suitable indication to a medical professional that a specific portion of the puncturing wire 18 is before the medical professional. For instance, the markings M1, M2, and M3 may be colors or indentations in the puncturing wire 18. In some examples in which the markings are colors, one or more of the markings may be a different color than one or more of the other markings to increase the visible distinction between the markings. For instance, the marking M1 may be red and the markings M2 and M3 may be black. In another instance, the marking M1 may be red, the marking M2 may be blue, and the marking M3 may be black.

In some aspects of the present disclosure, the puncturing wire 18 may be constructed of an inner core 30 (FIG. 1B) enclosed by an outer sheath 40 (FIG. 1C), such as the example puncturing wire 50 in FIG. 1C. As shown in FIG. 1B, the inner core 30 extends from a sharp cutting end 34 to a distal end 32. The sharp cutting end 34 may, for example, be constructed in accordance with the description pertaining to the sharp cutting end 20 above. As shown in FIG. 1C, the outer sheath 40 extends from a proximal end 44 to a dull gripping end 42. The dull gripping end 42 is adapted to be gripped by a medical professional while performing a PDT procedure. As shown in FIG. 1C, the sharp cutting end 34 of the inner core 30 is not enclosed by the outer sheath 40 so that the sharp cutting end 34 may puncturing a patient's trachea. Accordingly, the outer sheath 40 may enclose the inner core 30 from the distal end 32 of the inner core 30 to a portion of the inner core 30 prior to the sharp cutting end 34. The portion of the inner core 30 enclosed by the outer sheath 40 is depicted by the dashed lines within the outer sheath 40. Additionally, in such aspects in which the puncturing wire is constructed of an inner core 30 and an outer sheath 40, the outer sheath 40 may, in some instances, have the one or more markings as described above in connection with the puncturing wire 18.

The outer sheath 40 may protect the inner core 30 from external conditions, for example, frictional forces with a bronchoscope channel, and may provide additional durability and strength to the inner core 30 to prevent it from breaking. The outer sheath 40 may also provide extra rigidity, and accordingly strength, to the puncturing wire 50 to assist the puncturing wire 50 in puncturing a patient's trachea from within the trachea. In some instances, the outer sheath 40 may be coated with a layer of friction reducing material, such as polytetrafluoroethylene, to increase the ability of the puncturing wire 50 to slide within a bronchoscope channel or a patient's insides (e.g., the patient's air passage) during a procedure. In various aspects, the outer sheath 40 may be constructed in a way, and of a material, that makes the outer sheath 40 resistant to kinking. Accordingly, the puncturing wire 50 in such examples may be flexible to bend without its outer sheath 40 kinking, as depicted by the various configurations of the gripping end 22 of the puncturing wire 50 in FIG. 1C.

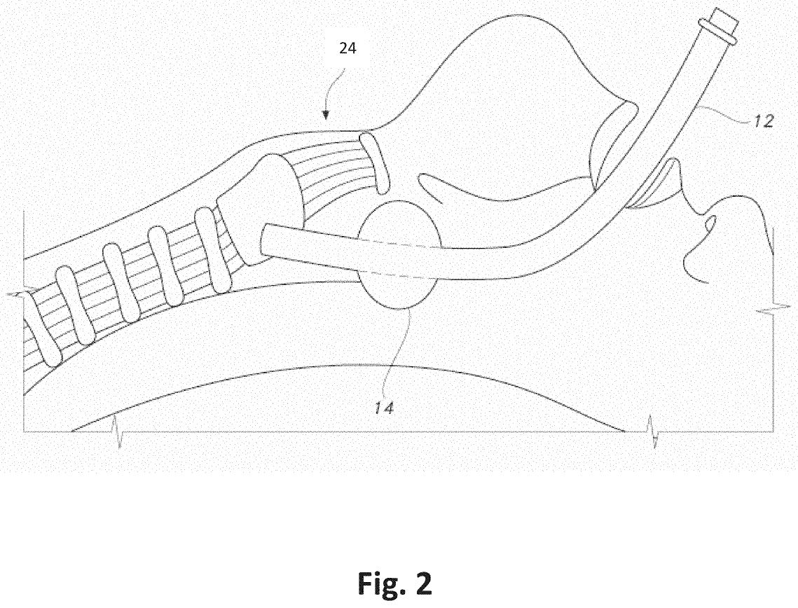

FIGS. 2 to 4 illustrate a PDT procedure according to an example of the present disclosure. The figures illustrate the procedure performed with the example puncturing wire 18; however, the procedure may also be performed with the example puncturing wire 50 or any other suitable puncturing wire consistent with the present disclosure. In various examples, two medical professionals may perform the procedure at a patient's bedside while the patient's vitals are monitored. Before starting the procedure, in some examples, the patient may undertake a pre-oxygenation cycle with 100% oxygen for a period of time (e.g., five minutes). In some instances, local anesthesia and muscle relaxant may be used during the procedure. The patient, as illustrated, may be in a supine position with the patient's head up and titled with a pillow or other object under the shoulders to hyperextend the neck.

The procedure may begin, as depicted in FIG. 2, by inserting an insertion end of a tracheal tube 12 through a patient's mouth into the patient's trachea. In various examples, the tracheal tube 12 may have an inflatable cuff 14 that is deflated as the tracheal tube 12 is inserted. In such examples, the tracheal tube 12 is positioned in the patient's trachea such that the deflated inflatable cuff 14 is just below the vocal chords 24, and then the inflatable cuff 14 is inflated, as depicted in FIG. 2, to provide a good seal and secure the tracheal tube 12 in place for the procedure.

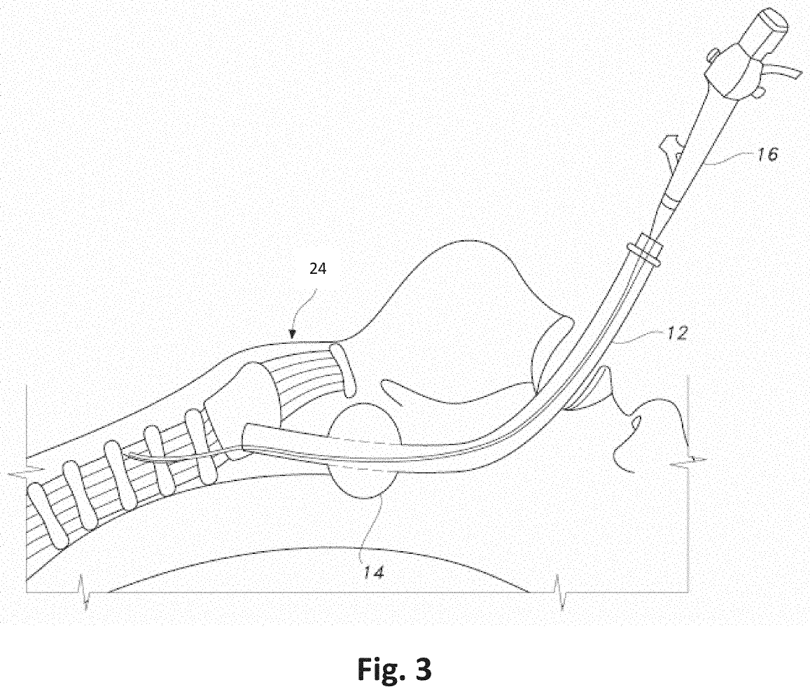

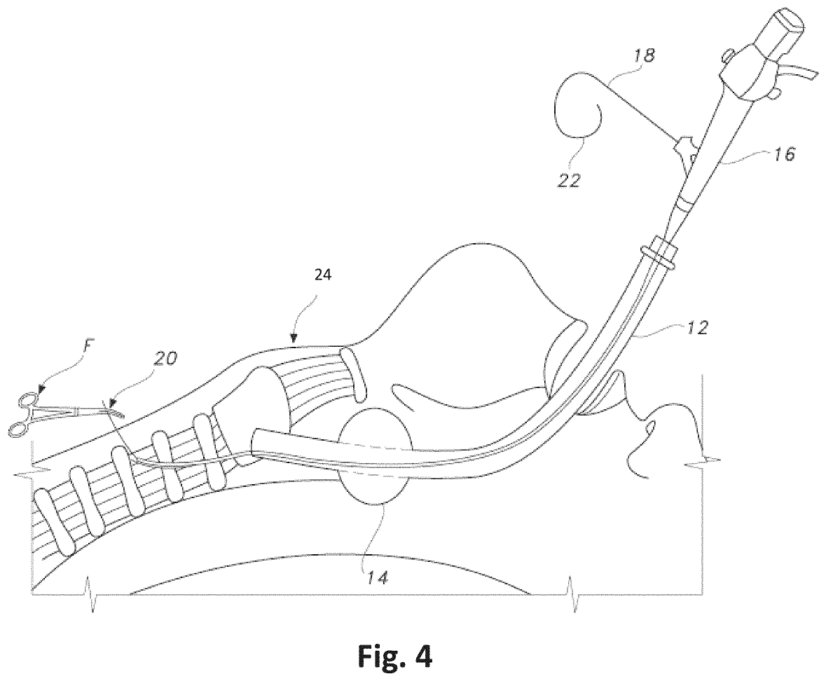

As depicted in FIG. 3, a bronchoscope 16 may then be advanced through the free end of the tracheal tube 12 until the patient's tracheal rings are located. FIG. 4 depicts that the puncturing wire 18 may then be advanced through a channel in the bronchoscope 16 until the cutting end 20 reaches a selected location in the trachea. Advancing the puncturing wire 18 within the bronchoscope 16 channel enables a medical professional to visualize both the patient's tracheal rings and the puncturing wire 18 with the bronchoscope 16, and accordingly properly position the puncturing wire 18 at the selected location. For example, in some instances the selected location may be between the second and third tracheal rings, as illustrated in FIG. 4.

As further depicted in FIG. 4, the sharp cutting end 20 of the puncturing wire 18 may puncture the patient's trachea from within the trachea at the selected location and pass through the patient's skin to create a stoma. The bronchoscope 16 may give the flexible puncturing wire 18 additional strength to puncture the trachea from within the patient. For example, the bronchoscope 16 provides additional rigidity to the flexible puncturing wire 18 and thus provides support when the medical professional manipulates the bronchoscope 16 and puncturing wire 18 to apply force to the patient's trachea with the sharp cutting end 20. The force causes the sharp cutting end 20 to puncture the patient's trachea and advance out of the patient.

Once the sharp cutting end 20 passes out of the skin, it may be retrieved by a forceps F or other suitable tool to prevent accidental lacerations of the medical professional by the sharp cutting end 20. A medical professional may then pull the puncturing wire 18 using the forceps F to advance the puncturing wire 18 out of the bronchoscope 16 and even further to a certain point. For instance, the medical professional may pull the puncturing wire 18 such that the marking M3 appears out of the patient's skin, the marking M2 appears, and then may stop when the marking M1 appears. At this point in the procedure, the medical professional may manipulate the puncturing wire 18 so that the gripping end 22 faces the patient's tracheal bifurcation. The medical professional may do so by using the bronchoscope 16 to position the gripping end 22. The medical professional may then advance the puncturing wire 18 back into the patient a certain amount. For instance, the medical professional may advance the puncturing wire 18 until the marking M2 is near the patient's skin, for example, when the marking M2 is just outside the patient or just within the patient.

Next in the procedure, a medical professional may then dilate the stoma with at least one dilator by passing the dilator over the puncturing wire 18. In some instances, an additional skin incision may be made to facilitate dilator passage if the stoma created by the sharp cutting end 20 is not sufficient. In some examples, more than one dilator may be used. For instance, a medical professional may pass a first, smaller dilator, such as a conventional 14-French dilator, over the puncturing wire 18 towards the patient's trachea to dilate the patient's skin and superficial tissues. The medical professional may then pass a second, larger dilator over the puncturing wire 18.

A medical professional may then remove the one or more dilators and may pass a tracheostomy tube obturator and a tracheostomy tube over the puncturing wire 18 to insert into the patient. The medical professional may then remove the puncturing wire 18 by pulling it all the way out of the patient. The medical professional may then perform endotracheal suction and establish ventilation for the patient through the tracheostomy tube. Accordingly, the disclosed PDT technique eliminates the need for a trocar and any complications that may result from using a trocar.

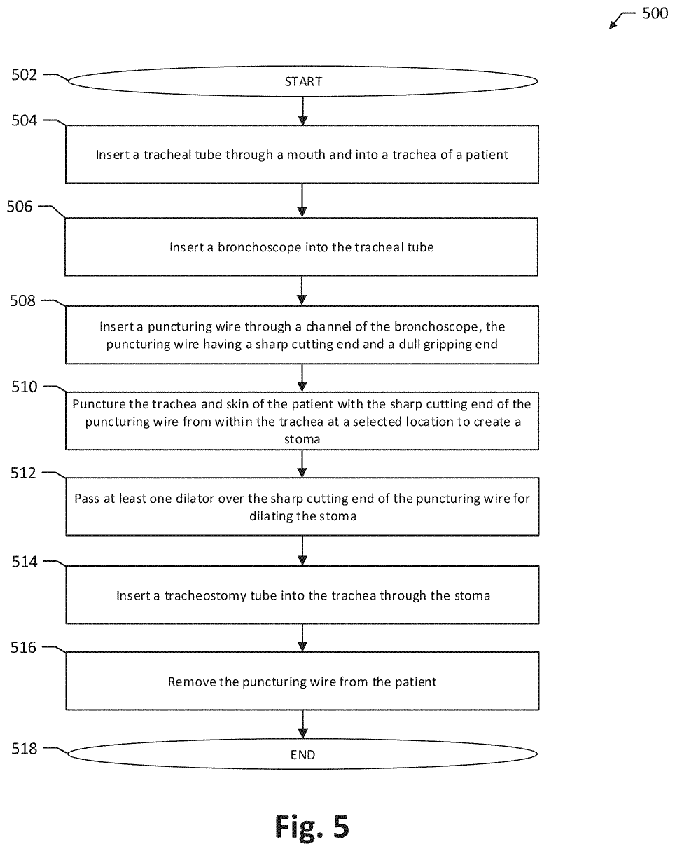

FIG. 5 illustrates an example method 500, according to an aspect of the present disclosure. Although the examples below are described with reference to the flowchart illustrated in FIG. 5, many other methods of performing the acts associated with FIG. 5 may be used. For example, the order of some of the blocks may be changed, certain blocks may be combined with other blocks, one or more of the blocks may be repeated, and some of the blocks described may be optional. At step 502, the method 500 begins. At step 504, a tracheal tube (e.g., the tracheal tube 12) is inserted through a patient's mouth and into the patient's trachea. At step 506, a bronchoscope (e.g., bronchoscope 16) is inserted into the tracheal tube. At step 508, a puncturing wire (e.g., the puncturing wire 18 or 50) is inserted through a channel of the bronchoscope. The puncturing wire has a sharp cutting end and a dull gripping end.

At step 510, the patient's trachea and skin are punctured with the sharp cutting end of the puncturing wire (e.g., the sharp cutting end 20 or 34) from within the trachea at a selected location to create a stoma. At step 512, at least one dilator is passed over the sharp cutting end of the puncturing wire for dilating the stoma. At step 514, a tracheostomy tube is inserted into the trachea through the stoma. At step 516, the puncturing wire is removed from the patient. At step 518, the example method 500 ends.

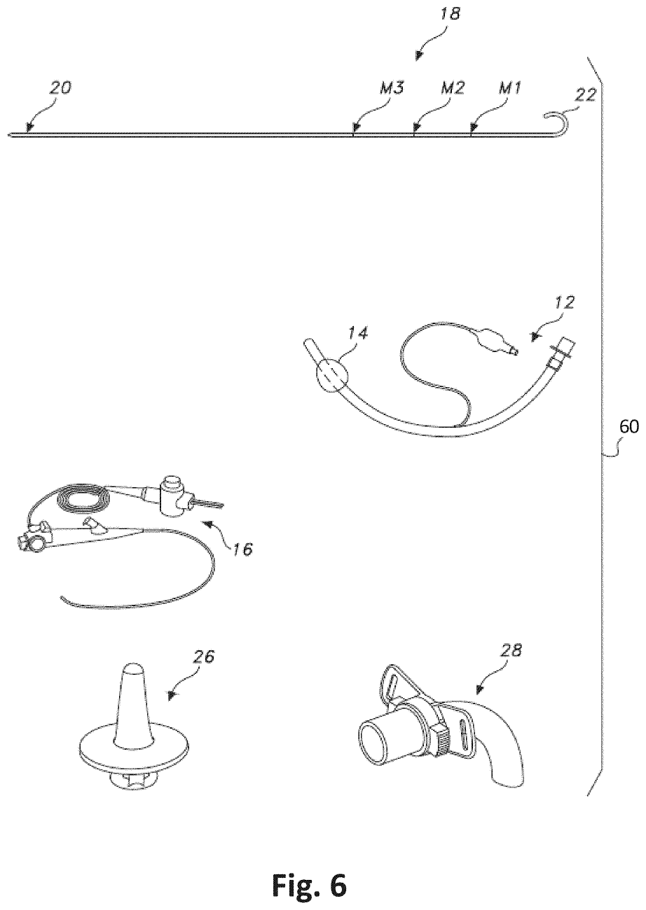

FIG. 6 illustrates an example kit 60 that may be used to implement the above-described techniques and methods for performing a PDT. In some aspects of the present disclosure, the example kit 60 may include a bronchoscope 16, a tracheal tube 12 with an inflatable cuff 14, and a puncturing wire 18. As described above, the puncturing wire 18 includes a sharp cutting end and a dull gripping end. The puncturing wire is designed to be removably inserted in a bronchoscope channel and the sharp cutting end is designed to cut through a patient's trachea from within the trachea. In some aspects of the present disclosure, the kit 60 may also include at least one stomal dilator 26 and a tracheostomy tube 28 in addition to the above described elements.

In other aspects of the present disclosure, the example kit 60 may additionally, or alternatively to the puncturing wire 18, include a puncturing wire 50. As described above, the puncturing wire 50 includes an inner core 30 at least partially enclosed by an outer sheath 40. The inner core 30 includes a sharp cutting end 34 and the outer sheath includes a dull gripping end 42 opposite the sharp cutting end 34 of the inner core 30. The puncturing wire 50 is constructed to be advanced through a channel in the bronchoscope 16 and the sharp cutting end 34 of the puncturing wire 50 is constructed to puncture a patient's trachea and skin from within the patient's trachea. In such other aspects, the kit 60 may also include at least one stomal dilator 26 and a tracheostomy tube 28.

Without further elaboration, it is believed that one skilled in the art can use the preceding description to utilize the claimed inventions to their fullest extent. The examples and embodiments disclosed herein are to be construed as merely illustrative and not a limitation of the scope of the present disclosure in any way. It will be apparent to those having skill in the art that changes may be made to the details of the above-described embodiments without departing from the underlying principles discussed. In other words, various modifications and improvements of the embodiments specifically disclosed in the description above are within the scope of the appended claims. For example, any suitable combination of features of the various embodiments described is contemplated. The scope of the invention is therefore defined by the following claims.

The invention is claimed as follows:

1. A method of performing a percutaneous dilational tracheostomy (“PDT”), comprising the steps of:
inserting a tracheal tube including an inflatable cuff through a mouth and into a trachea of a patient;
inserting a bronchoscope into the tracheal tube;
inserting a puncturing wire through a channel of the bronchoscope, wherein the puncturing wire includes a sharp cutting end opposite a dull gripping end;
puncturing the trachea of the patient with the sharp cutting end of the puncturing wire from within the trachea at a selected location to create a stoma;
passing at least one dilator over the sharp cutting end of the puncturing wire for dilating the stoma;
inserting a tracheostomy tube into the trachea through the stoma; and
removing the puncturing wire from the patient.

2. The method of performing a PDT as recited in claim 1, further comprising pulling the sharp cutting end of the puncturing wire once the sharp cutting end passes through at least the trachea or skin of the patient, wherein the pulling advances a portion of the puncturing wire out of the patient.

3. The method of performing a PDT as recited in claim 2, wherein the puncturing wire includes a plurality of markings, and wherein the sharp cutting end is pulled until at least one of the plurality of markings appears outside of the patient.

4. The method of performing a PDT as recited in claim 3, wherein the method further comprises manipulating the puncturing wire once a marking nearest the dull gripping end of the plurality of markings appears outside of the patient, wherein the manipulating includes positioning the puncturing wire such that the dull gripping end faces a tracheal bifurcation of the patient.

5. The method of performing a PDT as recited in claim 4, wherein the method further comprises, after the puncturing wire is manipulated, advancing the puncturing wire into the patient until the next adjacent marking to the marking nearest the dull gripping end is near the skin of the patient.

6. The method of performing a PDT as recited in claim 1, wherein the selected location is between the second and third tracheal rings of the patient.

7. The method of performing a PDT as recited in claim 1, wherein puncturing the trachea of the patient includes providing support to the sharp cutting end with the bronchoscope when the sharp cutting end punctures the trachea.

8. The method of performing a PDT as recited in claim 1, wherein the puncturing wire includes an inner core at least partially enclosed by an outer sheath, wherein the inner core includes the sharp cutting end, and wherein the outer sheath includes the dull gripping end opposite the sharp cutting end.

9. The method of performing a PDT as recited in claim 1, the method further comprising inflating the inflatable cuff of the tracheal tube at a position just below the vocal chords of the patient.

10. A kit for performing a percutaneous dilational tracheostomy (“PDT”), the kit comprising:
a bronchoscope including a channel;
a tracheal tube including an insertion end and a free end with an inflatable cuff near the insertion end, wherein the tracheal tube is adapted to receive the bronchoscope; and
a puncturing wire including a sharp cutting end and a dull gripping end, wherein the puncturing wire is adapted to be removably inserted in the channel of the bronchoscope, and wherein the sharp cutting end is adapted to cut a stoma through a trachea of a patient from within the trachea at a selected site.

11. The kit for performing a PDT as recited in claim 10, wherein the kit further comprises:
at least one stomal dilator; and
a tracheostomy tube.

12. The kit for performing a PDT as recited in claim 11, wherein the at least one stomal dilator includes a 14-French dilator.

13. The kit for performing a PDT as recited in claim 10, wherein the puncturing wire is coated in a friction-reducing material.

14. The kit for performing a PDT as recited in claim 13, wherein the friction-reducing material includes polytetrafluoroethylene.

15. The kit for performing a PDT as recited in claim 10, wherein the puncturing wire has a length between approximately 90 cm and approximately 1.5 m.

16. The kit for performing a PDT as recited in claim 10, wherein the puncturing wire includes at least one marking.

17. The kit for performing a PDT as recited in claim 16, wherein each of the at least one marking is a color or an indentation.

18. The kit for performing a PDT as recited in claim 10, wherein the puncturing wire includes a plurality of markings including a first marking positioned approximately 5 cm from the dull gripping end, a second marking positioned approximately 15 cm from the dull gripping end, and a third marking positioned approximately 20 cm from the dull gripping end.

19. The kit for performing a PDT as recited in claim 18, wherein the first marking is a first color, the second marking is a second color, and the third marking is a third color, and wherein the first color is different than at least one of the second color and the third color.

20. The kit for performing a PDT as recited in claim 10, wherein the puncturing wire includes an inner core at least partially enclosed by an outer sheath, wherein the inner core includes the sharp cutting end, and wherein the outer sheath includes the dull gripping end opposite the sharp cutting end.
